(19) United States
(12) Patent Application Publication
(10) Pub. No.: US 2005/0215958 A1
(43) Pub. Date: Sep. 29, 2005

(54) Aspiration device

(76) Inventor: Matthew Hawthorne, Hendra, Brisbane, Queensland (AU)

Correspondence Address: MICHAEL BEST & FRIEDRICH, LLP, 100 E WISCONSIN AVENUE, MILWAUKEE, WI, 53202, US

(21) Appl. No.: 10/504,797

(22) PCT Filed: Feb. 14, 2003
(86) PCT No.: PCT/AU03/00190

§ 371 Date: May 10, 2005

(30) Foreign Application Priority Data
Feb. 14, 2002 (AU) PS 0539

(51) Int. Cl. A61M 5/315

(52) U.S. Cl. 604/227

(57) ABSTRACT

An aspiration device (3) comprises a syringe barrel coupling element (50), for coupling to a barrel of a syringe (5), and a plunger coupling element (50), for coupling to a plunger of a syringe (8). The plunger coupling element (12) and the barrel coupling element (50) are moveable relative to each other by application of force to pressure portions (20, 60) associated with each element and the pressure portions (20, 60) are adapted to be forced towards each other by a user placing a finger tip (43) on a first pressure portion and a thumb tip (41) on a second pressure portion. Forcing the pressure portions (20, 60) towards each other forces the plunger (8) to move out of the barrel (5), effecting an aspiration mode.

### FIELD OF THE INVENTION

The present invention relates to an aspiration device and especially but not exclusively to an aspiration device in the form of a disposable holder which can be attached to a disposable syringe in order to facilitate the extraction of tissue and/or fluid samples by the technique of fine needle aspiration.

### BACKGROUND OF THE INVENTION

Fine needle aspiration typically involves the extraction of samples of tissue through a small needle. Typically, this is performed by inserting the needle of a syringe into the tissue to be sampled, moving the needle in the tissue and applying suction by pulling the plunger of the syringe with one hand while holding the syringe barrel with the other hand. Syringe holding devices for use with fine needle aspiration are known and include devices which are expensive and reusable such as the pencil grip type syringe holder of U.S. Pat. No. 5,830,152, which includes a spring to provide a biasing force which tends to force the plunger outwardly relative to the syringe barrel and a catch for restraining the action of the spring. The syringe holder can be operated in an aspiration mode with a single hand as a button is provided allowing a user to deactivate and reactivate the catch and thus regulate aspiration. An alternative reusable device is a “pistol grip” device, as described in U.S. Pat. No. 4,850,979 (incorporated as herein by reference) which adopts a pistol-like configuration in which the syringe is positioned between a handle of the device and the tissue to be sampled. A disadvantage of this device is that the hand used to operate the device is a considerable distance away from the tissue to be sampled, making needle placement and control difficult.

Reusable aspiration devices have a further disadvantage in that sterilising by way of auto-claving is required in order to guard against transmission of diseases transmitted via bodily fluids.

Disposable aspiration devices have been disclosed in U.S. Pat. No. 4,594,073 and U.S. Pat. No. 5,115,816. These involve use of a pistol-like grip in which a user engages a handle portion with the base of the thumb or palm of the hand and a trigger portion with one or more fingers, and squeezes the fingers towards the palm in order to operate the device in an aspiration mode.

It is considered that such disposable devices suffer from a difficulty in the positioning thereof and in accurately providing the required amount of pressure to provide aspiration, under certain circumstances.

### SUMMARY OF THE INVENTION

According to a first aspect of the present invention, there is provided an aspiration device comprising:
a syringe barrel coupling element, adapted in use to be coupled to a barrel of a syringe, and to remain substantially fixed relative to said barrel; and
a plunger coupling element, adapted in use to be coupled to a plunger of a syringe and to move relative to said barrel coupling element so that forced relative motion of said plunger coupling element and said barrel coupling element forces relative motion of the syringe plunger and syringe barrel;
wherein the plunger coupling element and the barrel coupling element are moveable relative to each other by application of force to at least one pressure portion associated with each element; and
said pressure portions associated with the elements are adapted to be forced towards each other by a user placing a finger tip on a first of said pressure portions and a thumb tip on a second of said pressure portions and wherein forcing said first and second pressure portions generally towards each other forces the plunger to move substantially axially in an outward direction relative to the barrel.

This constitutes an aspiration mode of the aspiration device.

The barrel coupling element may be formed integrally with a barrel of a syringe.

Preferably, the aspiration device is a holder for a syringe.

Preferably, said first pressure portion is substantially rigidly coupled to the plunger coupling element.

Preferably, said first pressure portion constitutes part of the plunger coupling element.

Preferably, said second pressure portion is substantially rigidly coupled to the barrel coupling element.

Preferably, said second pressure portion constitutes part of the barrel coupling element.

Preferably, said first pressure portion is adapted to receive and be forced by an index-finger finger tip of a user. In this case the first pressure portion constitutes a finger tip pressure portion.

Preferably, said first pressure portion is adapted to receive and be forced by an index-finger finger tip and a second-finger finger tip of a user.

Preferably, the first pressure portion includes at least one finger tip receiving area.

Preferably, the at least one finger tip receiving area is dimensioned to receive a substantial portion of a finger tip pad of a user.

Preferably, the or each finger tip receiving area provides a surface for contact with a finger tip of a user, the surface being oriented in a plane generally perpendicular to an axis of the aspiration device and/or syringe.

Preferably, a surface of the or each finger tip receiving area is dimensioned to be approximately similar in length and width. A preferred surface shape is a concave part-spherical surface, preferably formed as a depression in the pressure portion.

Preferably, said first pressure portion includes. two finger tip receiving areas.

Preferably, said two finger tip receiving areas have centres which are, in use, substantially equidistant from a longitudinal axis of said device and/or of a syringe coupled to the device in use.

Preferably, the first pressure portion and the second pressure portion are arranged, in use, so that an imaginary line substantially coplanar, and more preferably substantially parallel, to the longitudinal axis of the device and/or of a syringe coupled to the device in use, passes through both the first and second pressure portions.

Preferably, the first and second pressure portions are arranged, in use, so that an imaginary line which passes through a point located generally centrally between the two finger tip receiving areas and which. passes through a generally central point of the part:of the second pressure portion which is adapted to receive a thumb tip, would be substantially coplanar, and more preferably parallel, to the longitudinal axis of the device and/or of a syringe coupled to the device in use. The line may be coplanar with, rather than parallel to, the syringe axis if, for example, the finger tip receiving areas are diametrically opposed with respect to the syringe.

Preferably, said first pressure portion is provided with finger tip retaining means to retain one or more finger tips of a user.

Preferably, said finger tip retaining means provides a retaining surface against which, in use, one or more backs of the one or more finger tips may press.

Preferably, the second pressure portion is adapted to receive and be forced by a thumb tip of a user. In this case the second pressure portion constitutes a thumb tip pressure portion.

Preferably, said second pressure portion is provided with thumb tip retaining means to retain a thumb tip of a user.

Preferably, said thumb tip retaining means provides a retaining surface against which, in use, the back of the thumb tip may press.

Preferably, the device is adapted to allow a user to move said first and second pressure portions away from each other by pressing the back of the thumb tip against the retaining surface of the thumb tip retaining means and the back or backs of the one or more finger tips against the retaining surface of the finger tip retaining means and moving apart said thumb tip from said one or more finger tips. This constitutes an expressing or injection mode of the aspiration device.

Preferably, said finger tip retaining means includes adjustment means. The adjustment means is to allow for retention of finger tips of different sizes.

Preferably, said finger tip retaining means adjustment means comprises an adjustment member which is selectively locatable in adjustment member retaining means coupled to the pressure portion.

Preferably, said adjustment member of the finger tip retaining means includes a number of teeth spaced along the length thereof and said adjustment member retaining means includes a pawl portion adapted to engage one or more of said teeth.

Preferably, said thumb tip retaining means includes adjustment means. The adjustment means is to allow for retention of thumb tips of different sizes.

Preferably, said thumb tip retaining means adjustment means comprises an adjustment member which is selectively locatable in adjustment member retaining means coupled to the pressure portion.

Preferably, said adjustment member of the thumb tip retaining means includes a number of teeth spaced along the length thereof and said adjustment member retaining means includes a pawl portion adapted to engage one or more of said teeth.

Preferably, said plunger coupling element includes a plunger flange receiving portion and includes a first connection portion between the plunger flange receiving portion and the pressure portion of the plunger coupling element.

Preferably, said barrel coupling element includes a barrel flange receiving portion, and includes a second connection portion between the barrel flange receiving portion and the pressure portion of the barrel coupling element.

Preferably, said first and second connection portions are shaped so that, in use, the plunger coupling element and barrel coupling element are slidably coupled.

Preferably, in use, at least part of one of the connection portions is retained in at least part of the other connection portion.

Preferably, one of the connection portions includes a channel which, in use, accommodates at least part of the other connection portion.

Preferably, the channel includes a deeper wider portion and a shallower narrower portion.

The channel may be provided in the barrel engaging element.

In this case, the plunger engaging element preferably includes a rod portion which fits into the channel and cooperates therewith.

The rod portion of the plunger engaging element may be positioned in the channel, on assembly of the device and the plunger flange receiving portion may be attached to the rod portion after it is positioned in the channel.

According to a second aspect of the present invention, there is provided an aspiration device which allows aspiration and injection modes to be operated using a single hand and with the hand being in substantially the same position relative to the device in both aspiration and injection modes.

Preferably, said device includes a finger tip pressure portion.

Preferably, said finger tip pressure portion is provided with finger tip retaining means to retain one or more finger tips of the user.

Preferably, said device includes a thumb tip pressure portion.

Preferably, said thumb tip pressure portion is provided with thumb tip retaining means to retain a thumb tip of a user.

Preferably, said finger tip retaining means provides a retaining surface against which, in use, one or more backs of the one or more finger tips may press.

Preferably, said thumb tip retaining means provides a retaining surface against which, in use, the back of the thumb tip may press.

Preferably, forcing said finger tip pressure portion and said thumb tip pressure portion towards each other operates the aspiration mode of the device.

Preferably, forcing the finger tip pressure portion and the thumb tip pressure portion away from each other, most preferably by pressure of the back of at least one finger tip on said finger tip retaining surface and the back of the thumb on the thumb tip retaining surface, operates the injection mode of the device. The device may be a holder for a syringe. Alternatively, at least part of the device may be formed integrally with a syringe.

Preferably, said finger tip pressure portion is, in use, coupled substantially rigidly to a plunger of a syringe.

Preferably, the thumb tip pressure portion is, in use, coupled substantially rigidly to a barrel of the syringe.

It will be appreciated that many or all of the preferred and/or essential features of the first aspect of the present invention may be incorporated, as preferred features, in devices in accordance with the second aspect.

### BRIEF DESCRIPTION OF THE DRAWINGS

Embodiments of aspiration devices in accordance with the invention will now be described, by way of example only, with reference to the accompanying drawings in which:

FIG. 1 is a side elevation of a plunger coupling element of an embodiment of an aspiration device;

FIG. 2 is a partial cross-sectional view on II-II of FIG. 1;

FIG. 3 is a side elevation of a barrel coupling element of an embodiment of an aspiration device;

FIG. 4 is a partial cross-section on IV-IV of FIG. 3;

FIG. 5 is a side elevational view, with some internal details shown, of an embodiment of an aspiration device;

FIG. 6 is a bottom view corresponding to FIG. 5;

FIG. 7 is a partial cross-sectional view on VII-VII of FIG. 5;

FIG. 8 is a partial cross-sectional view illustrating assembly of part of the plunger coupling element of the embodiment of FIGS. 1 to 7;

FIGS. 9 and 10 are perspective views of the embodiment of FIGS. 1 to 8;

FIGS. 11 and 12 are perspective views of the embodiment of FIGS. 1 to 10 illustrating the positioning of the hand of a user relative to the device;

FIG. 13 is a perspective view of an alternative embodiment;

FIGS. 14(a) and 14(b) are partial cross-sectional views of parts of FIG. 13 before assembly;

FIGS. 15(a) and 15(b) show the parts illustrated in FIGS. 14(a) and 14(b) after assembly;

FIG. 16(a) is a plan view of an element of an alternative embodiment; and

FIGS. 16(b) and 16(c) are cross-sections on b-b and c-c, respectively, of FIG. 16(a).

### DETAILED DESCRIPTION OF THE DRAWINGS

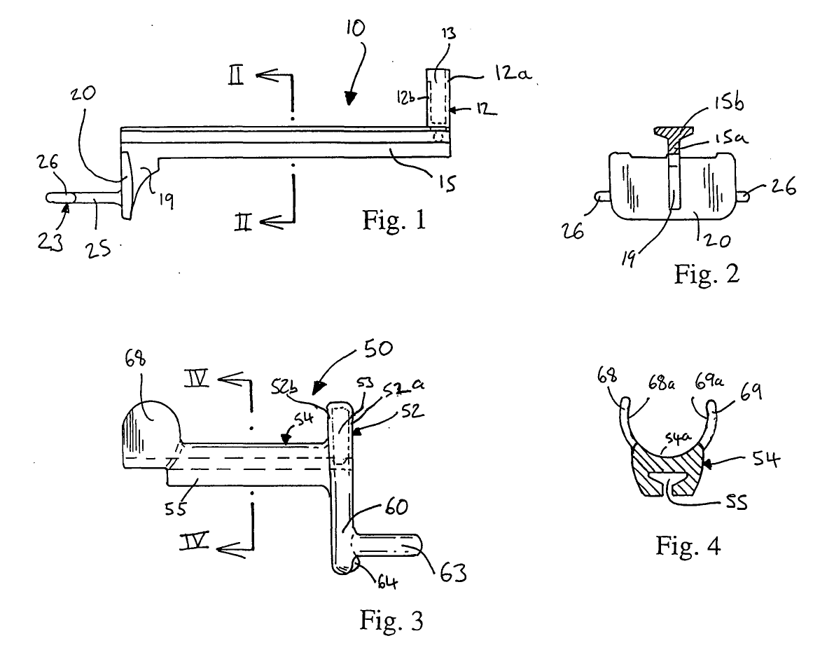

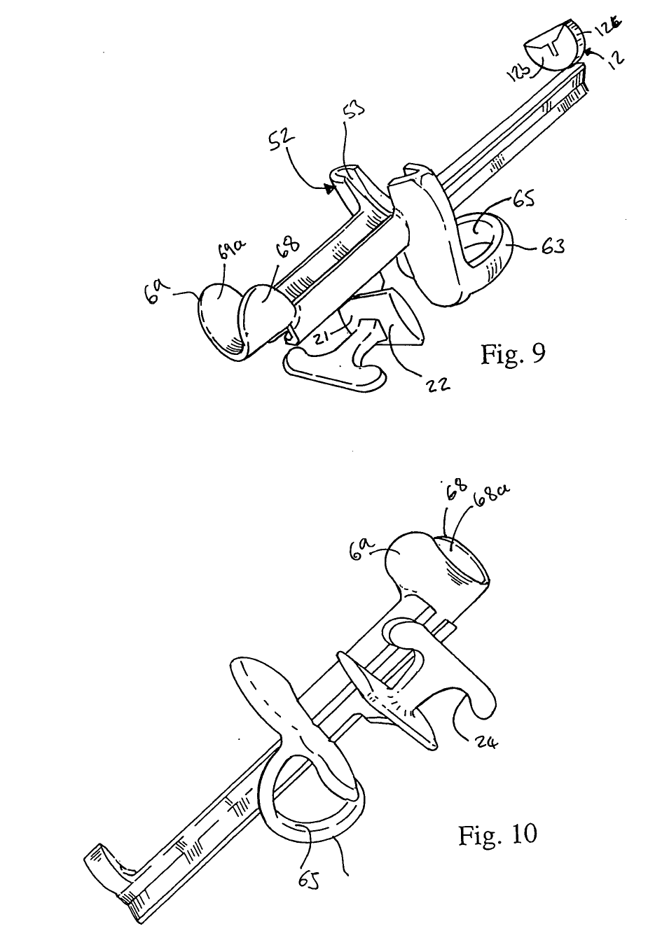

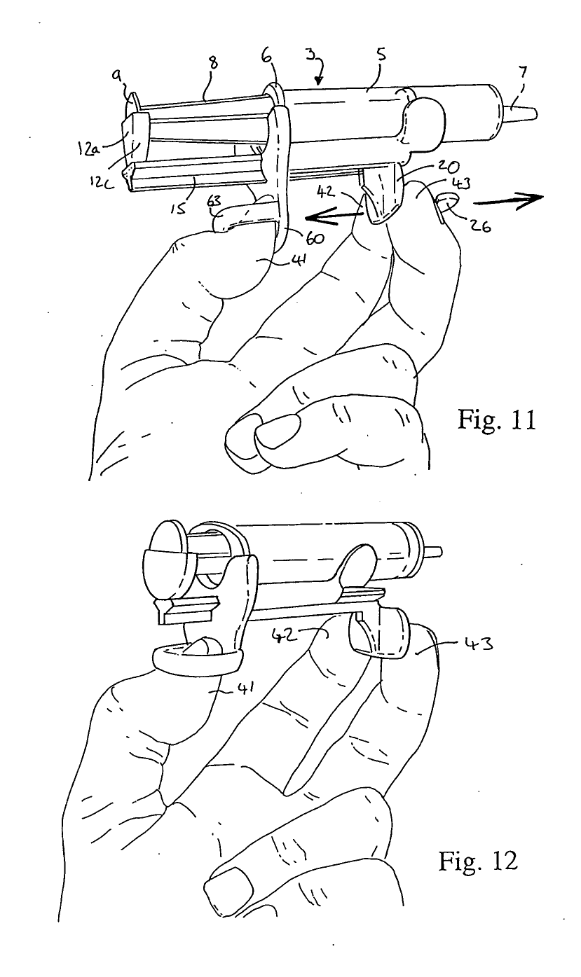

With reference to FIGS. 1 to 12, an embodiment of an aspiration device in accordance with the present invention comprises a plunger coupling element, generally designated 10, and shown in side elevation in FIG. 1, and a barrel coupling element, generally designated 50, and shown in side elevation in FIG. 3. In use, as shown in FIGS. 11 and 12, the plunger coupling element 10 and barrel coupling element 50 are assembled to form an aspiration device 1 and operatively attached to a syringe, generally designated 3. As shown in FIGS. 11 and 12, the syringe is of a disposable plastic type and includes a barrel 5 having a barrel flange 6 at a first end thereof and a hub portion 7 suitable for accepting a needle (not shown) at a second end thereof. The barrel provides an interior cavity. In use a plunger 8 is provided at a first end thereof with a resilient sealing member (not shown) which is moveable inside the interior cavity of the barrel. The plunger is provided at second end thereof with a plunger flange 9 which is operable by application of pressure by the thumb of a user in order to move the sealing member of the plunger axially in the cavity to express or inject fluid from the syringe cavity through the hub and needle attached thereto.

The plunger coupling element comprises a first connection portion in the form of a rod portion 15 which is provided with a plunger flange receiving portion in the form of a plunger flange cap 12 at one end thereof. The plunger flange cap 12 is generally semi-circular in form and has a generally semi-circular rear wall 12a and a generally semi-circular front wall 12b. The front and rear walls 12b, 12a are mutually connected at the curved edges thereof by a generally half-cylindrical side wall 12c, which is short in axial extent so that the distance between the front end rear walls is not great. The front, rear and half-cylindrical walls 12b, 12a, 12c of the plunger flange cap 12 define a generally semi-circular cavity 13 therein which is adapted to receive a plunger flange 9 of a syringe 3. In use, the plunger flange 9 is frictionally retained in the cavity 13 so that the plunger coupling element 10 is coupled to the plunger flange 9 and thus to the plunger 8 of the syringe. The front wall 12b may have a cut out portion in order to allow or facilitate accommodation of the plunger flange.

The rod portion 15 is generally T-shaped in cross-section, having a wider part 15a and a narrower part 15b as for example shown in FIG. 2. At the opposite end of the rod portion 15 to the plunger flange cap 12 is provided a finger tip pressure portion 20 which provides first and second finger tip receiving areas 21, 22 respectively, generally in a plane perpendicular to the axis of the rod portion 15. Each of the finger tip receiving areas 21, 22 provides a finger tip pressure surface generally in a plane perpendicular to the axis of the rod portion 15 (and, in use, generally perpendicular to the axis of the syringe). The finger tip pressure portion 20 is attached to and formed integrally with the rod portion 15 in this embodiment, for example from a plastics material by injection moulding. A support 19 extending between the finger tip pressure portion 20 and the narrower part 15b of the rod portion 15 is provided to add strength to the connection between the finger tip pressure portion 20 and the rod portion 15.

A finger tip retaining means 23 is attached to the finger tip pressure portion 20. The finger tip retaining means 23 is a generally T-shaped member and includes a shank portion 25 attached at a first end thereof to the finger tip pressure portion 20, and positioned thereon generally between the first and second finger tip receiving areas 21, 22. At a second end of the shank portion, there is provided a cross-piece 26 which extends generally parallel to the finger tip pressure portion on either side of the shank 25, and each arm thereof has a finger tip retaining surface 24 generally facing one of the first or second finger tip receiving areas 21, 22. In use, the finger tips of a user are retained between the cross-piece 26 of the finger tip retaining means 23 and the first and second finger tip receiving areas 21, 22 of the finger tip pressure portion 20. It is preferred, though not essential, that during use the finger tips should remain in contact both with the finger tip receiving areas 21, 22 and with the finger tip retaining means.

The barrel coupling element 50 comprises a second connection portion 54 which has an arcuate inner surface 54a adapted to engage an outer surface of the barrel 5 of the syringe 3 and which is elongate and has a elongate channel 55 provided therethrough. The elongate channel is adapted to receive the rod portion 15 of the plunger coupling element 10 and has a complementary shape, being generally T-shaped in cross-section, the bottom of the stem of the T forming a narrow elongate axial opening in a side of the second connection portion 54. At a first end of the elongate second connection portion 54 there is provided (extending upwardly as shown in the drawings) a barrel flange receiving portion 52 which includes spaced apart first and second walls 52a, 52b defining a barrel flange receiving cavity 53 therebetween. The barrel flange receiving cavity 53 is generally half annular and is adapted to receive a barrel flange 6 of a syringe 3 therein. In use, the barrel flange 6 is retained frictionally in the barrel flange receiving cavity 53 thus coupling the barrel coupling element to the barrel 5 of the syringe 3. The edges of the first and second walls 52a, 52b are arcuate in order to accommodate the syringe barrel which extends away from the barrel flange in one direction, and the syringe plunger, which in use extends away from the barrel flange in the other direction.

Continuous with the barrel flange receiving portion 52 and coplanar therewith, but, in use, further from the syringe, there is provided a thumb tip pressure portion 60 which includes an area (not shown) against which pressure may be applied by a user by pressing with a thumb tip 41 (as illustrated in FIGS. 11 and 12). A thumb grip 64 in the form of a small protrusion is provided towards the edge of the thumb tip pressure portion 60 in order to help prevent inadvertent sliding of a thumb from the thumb tip receiving area in use. Alternative or additional configurations serving the same function may be provided, such as a suitably convex shape for the thumb tip receiving area. Attached to the thumb tip pressure portion 60 (and, in the embodiment of FIGS. 1 to 12, formed as a single piece therewith) there is provided a thumb tip retaining means 63 in the form of a generally semi-circular loop attached at its first and 4 second ends to spaced apart points on the thumb tip pressure portion 60 in order to define a generally semi-circular space therebetween. In use, the generally semi-circular space has a thumb tip inserted therein and the thumb tip is retained when moving parallel to the axis of the second connection portion. The thumb tip retaining means 63 provides an inner surface of the semi-circular loop which constitutes a thumb tip retaining surface 65, and against which the back of a thumb tip may push in order to apply a force to the thumb tip pressure portion 60 via the ends of the semi-circular loop. Such a force, in use, is applied in the opposite direction to the force applied by pressing the front of a thumb tip upon the thumb tip pressure portion 60.

At the distal end of the second connection portion 54, there are provided first and second arcuate securement elements 68, 69 which in use extend partially around the barrel 5 of a syringe in order to help retain the barrel coupled to the barrel coupling element 50. The first and second arcuate securement elements 68, 69 each have respective inner barrel engaging surfaces 68a, 69a which are continuous with the arcuate surface 54a and which together form a surface which in use receives and contacts the outer surface of the barrel 5.

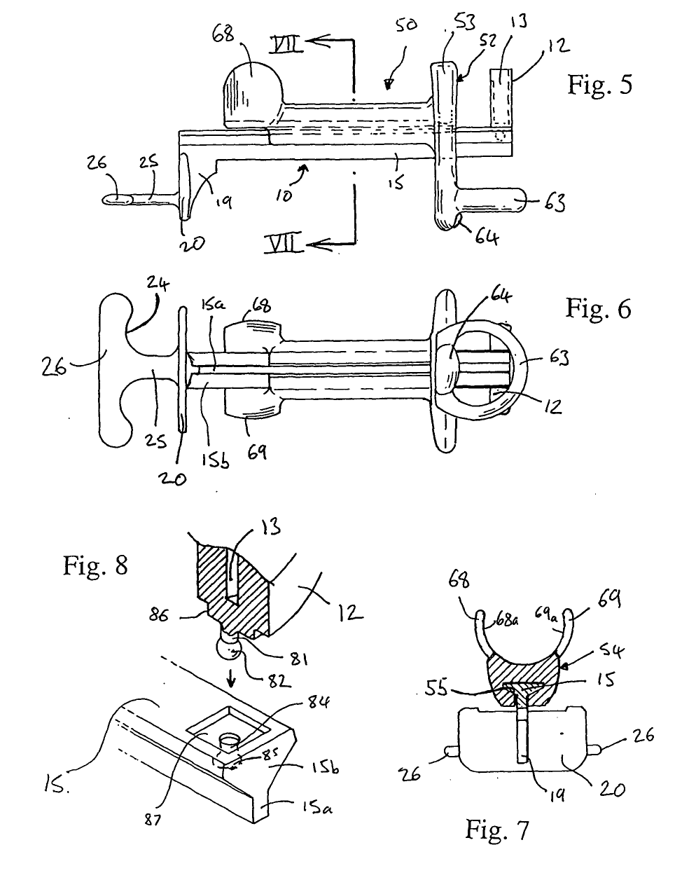

As shown in FIGS. 5 to 7 and 9 to 12, for use the plunger coupling element 10 and barrel coupling element 50 are assembled so that the rod portion 15 of the plunger coupling element is located in, and slidable within, the channel 55 of the barrel coupling element. Assembly is performed by merely sliding the rod portion 15 into the channel 55 by insertion of the first end of the rod portion 15 into the end of the channel 55 which is adjacent the first and second arcuate securement elements 68, 69. Because the plunger flange cap 12 would, if already attached to the rod portion 15 prevent such insertion, in this embodiment the plunger flange cap 12 is not attached to the rod portion 15 until after the rod portion has been positioned in the channel 55. It is envisaged that the plunger flange cap 12 could be attached to the rod portion 15 in any of a number of appropriate ways which would be evident to the person skilled in the art. For example, attachment could be merely by gluing with a suitably strong adhesive or a plastic welding substance.

FIG. 8 illustrates one means of connecting the plunger flange cap 12 to the rod portion 15. In this embodiment, the attachment is achieved by inserting a male connection element on the plunger flange cap 12 into a corresponding female connection element at an appropriate position of the rod portion 15. The male connection element in this embodiment comprises a short cylindrical stem portion 81 connected at a first end thereof to the plunger flange cap 12 and having a rounded protuberance 82 with greater diameter than the diameter of the cylindrical portion 81, at a second end thereof. The female connection element in the rod portion 15 comprises a correspondingly shaped cavity, which has a short cylindrical cavity portion 84 open at a first end thereof and widening at a second end thereof into a protuberance-accommodating cavity 85. The slightly resilient nature of plastics materials allows the protuberance 82 to be forced into the protuberance-receiving cavity 85 and to be retained therein thus coupling the plunger flange cap 12 and the rod portion 15. Further shaping of the areas of the plunger flange cap 12 and rod portion 15 may be provided in order to strengthen the coupling against the forces which will be applied thereto in use and, in the illustrated embodiment, such further shaping comprises an upstanding square portion 86 in the area immediately surrounding the cylindrical stem portion 81, and which is adapted to be received in a correspondingly shaped and positioned recess 87 provided in the rod portion 15.

An alternative means of coupling a plunger flange cap to a rod portion is illustrated in FIGS. 14a, 14b, 15a and 15b and will be discussed hereafter. Another alternative is to provide a male threaded member extending from the plunger flange cap and an aperture for receiving the male threaded member in the rod portion, in which case the aperture would be provided with a corresponding female thread, or alternatively the male member could extend out of the aperture on the distal side of the rod portion and be secured by a fixing nut with a suitable female thread. Such an arrangement might provide a more robust connection than those described with reference to FIGS. 8, 14a, 14b, 15a and 15b, but the “snap-in” arrangements described with reference to the aforementioned drawings are more convenient to assemble.

As shown in FIGS. 11 and 12, in use the aspiration device 1 is coupled to a syringe 3 and operated by the user placing an index-finger finger tip 42 and a second-finger finger tip 43 on the finger tip pressure portion 20 where they are retained against moving directly off the finger tip pressure portion 20 by the finger tip retaining surface (24, but not shown in FIGS. 11 and 12) of the cross piece 26 of the finger tip retaining means 23. The thumb tip 41 of the same hand of the user is placed upon the thumb tip pressure portion 60 where it is retained against movement directly away from the thumb tip pressure portion 60 by the thumb tip retaining means 63, and in particular by the thumb tip retaining surface (65, but not shown in FIGS. 11 and 12).

In use a needle (not shown) of the syringe is placed into tissue to be sampled, with the plunger inserted substantially fully into the barrel, so that the index and first fingers of the user are towards the end of the barrel 5 to which the needle is attached. This allows the needle to be inserted into the tissue with considerable precision since the fingers are near the tissue to be sampled. Since only one hand is required to operate the aspiration device, the other hand of the user can be used to steady the tissue to be sampled or provide tension to the skin thereof. Because the user's first and second fingers are close to the needle and spaced substantially equally from a central axis of the needle, manipulation of the device and needle is facilitated allowing greater control and reducing the likelihood of needle stick injuries to the user's other hand or other misadventure due to poor or erroneous positioning of the needle. The needle is then moved in the tissue, and again this operation may be performed with precision because of the proximity of the user's hand to the tissue. Tissue to be sampled is then aspirated by the user simply squeezing together the fingers and thumb of the hand operating the device thus exerting pressure on the finger tip and thumb tip pressure portions 20, 60, moving the plunger axially outwardly with respect to the barrel in order to provide aspiration. This constitutes an aspiration mode.

Because the device is operated by the finger tips and thumb tip of the user, the amount of pressure applied can be easily and precisely regulated. Aspiration is performed with the hand in a position, and performing an action, which will be familiar to a user who is familiar with normal operation of syringes, and this familiarity will tend to facilitate use and enhance accuracy in operation. It will also be appreciated that, in use, the two finger tips and the thumb tip of the user (and corresponding parts of the device) form a triangle, and the axis of the device or syringe, in use, is parallel to a line bisecting the triangle. Furthermore, in preferred embodiments the axis of the device or syringe lies in a plane which is orthogonal to the plane of the triangle and which includes the line bisecting the triangle. This allows a high degree of control over the syringe or device and, in particular, over the needle, which is coaxial with the axis of the device or syringe.

Aspiration can be halted by cessation of finger tip and thumb tip pressure, and if necessary further aspiration of fluid can be prevented by exertion of slight pressure by the user on the finger tip and thumb tip retaining surfaces 24, 65. The needle may then be removed from the tissue to be sampled, and the sample may be expressed by the user forcing apart the finger tips and thumb tip of the hand operating the device. This exerts pressure from the backs of the finger tips and thumb tip upon the finger tip retaining means and thumb tip retaining means and causes the plunger to move axially into the barrel 5 of the syringe. This constitutes an injection mode.

This embodiment therefore provides an aspiration device which can be used in both aspiration and injection modes with a single hand and with the hand of the user in substantially the same position relative to the device (and the syringe) in either mode. Although described above with reference to taking a tissue sample, for example, in fine needle aspiration, the device may be used for other functions where both injection and aspiration modes are desired, for example, in drawing a medication into a syringe and then injecting the medication into a patient. The embodiment may also be used in, for example, taking blood samples. After use, the aspirating device and syringe may be disposed of in an appropriate manner, since in this embodiment the aspiration device is intended as a disposable article, as are most syringes.

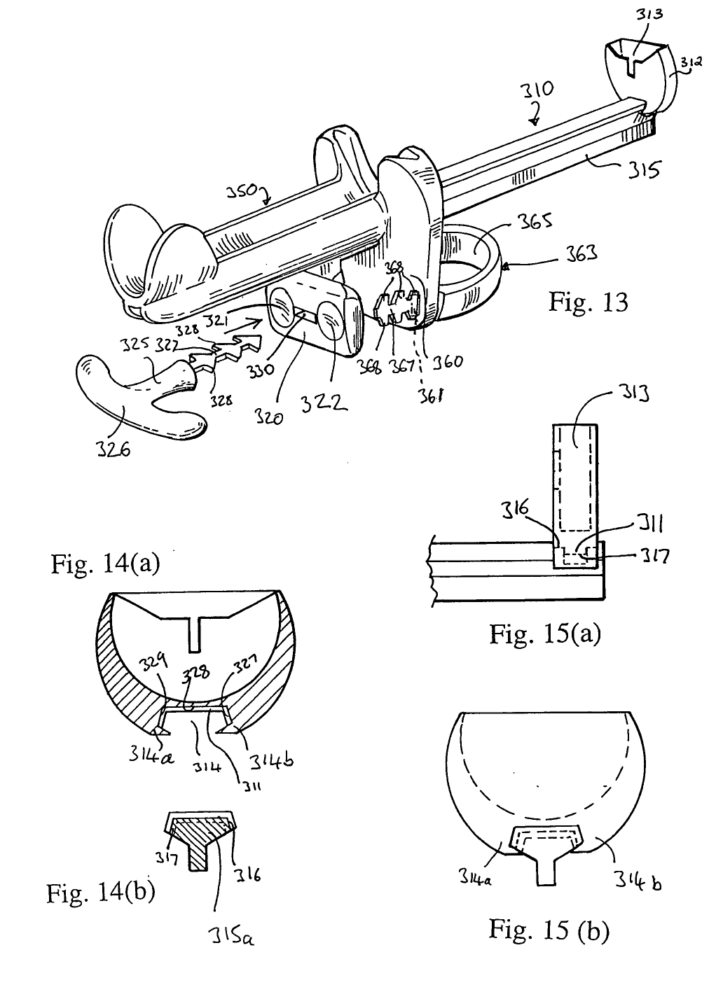

An alternative embodiment is shown in FIGS. 13, 14(a), 14(b), 15(a) and 15(b). With reference to FIG. 13, it can be seen that there are many similarities between this embodiment and the embodiment of FIGS. 1 to 12 and only the differences between the embodiments will be discussed in detail. In FIG. 13, a finger tip retaining means 323 comprises a shank portion 325 and a cross-piece 326 which provides a finger tip retaining surface (not shown) which is functionally similar to the finger tip retaining surface 24 of the embodiments of FIGS. 1 to 12. The finger tip retaining means 323 is illustrated in FIG. 13 detached from a finger tip pressure portion 320. The finger tip pressure portion 320 has a first finger tip receiving area 321 and a second finger tip receiving area 322 provided thereon each in the form of a slight concave depression. The finger tip pressure portion 320 also has a receiving aperture 330 provided approximately centrally between the first and second finger tip receiving areas 321, 322. In this embodiment, the first and second finger tip receiving areas 321, 322 are spaced apart on a substantial continuous finger tip pressure portion 320. Alternative embodiments are possible in which the finger tip pressure portion is discontinuous, for example with a finger tip retaining area at each of two portions diametrically opposed, in use, with respect to the axis of the syringe. The finger tip retaining means 323 has an adjustment member in the form of a stem portion 327 provided with a plurality of teeth 328 thereon. The teeth 328 and receiving aperture 330 are adapted to cooperate so that the stem 327 can be pushed into the receiving aperture 330 a desired distance and will be retained in that position (even if a force is applied to the finger tip retaining means biasing it away from the finger tip pressure portion 320) by retention of an appropriate one of the teeth 328 in the aperture.

Although the embodiment of FIG. 13 is shown as having large teeth 328 (equivalent to barbs on the stem) a preferred mechanism for fixing the stem 327 of the finger tip retaining means 323 to the finger tip pressure portion 320, is similar to that commonly used in fasteners known as cable ties, bundle ties or zip ties which include a head portion with a plurality of pawls provided therein and adapted to receive a strap portion with a plurality of transverse teeth provided thereon. In use, the strap portion may be inserted into the head portion in order to tighten the tie and the pawls and teeth interact in a ratchet-like manner in order to prevent loosening of the tie. Such a tie is described in U.S. Pat. No. 3,605,199 which is incorporated herein by reference, and the skilled person will appreciate that a mechanism of the type described can easily be adapted for use in the described embodiment. The features and connective function of the head of the tie would preferably be incorporated into the finger tip pressure portion, 320 and the features and connective function of the strap would preferably be incorporated into the stem of the finger retaining means 323. In this way, a conveniently adjustable finger tip retaining means is provided which can be adjusted to effectively retain the finger tips of a user and which provides a finger tip retaining surface enabling the plunger coupling element to be forced in one axial direction by pressure of the fronts of the finger tips against the finger tip receiving areas and to be moved in the opposite axial direction by pressure of the backs of the finger tips against finger tip retaining surfaces provided on the finger tip retaining means. Alternative means of adjustment of the finger and thumb tip retaining means may be incorporated, for example, adjustment members having straps with apertures spaced longitudinally may be retained by buckles or other retaining means having one or more elements which engage in one or more of the apertures.

As shown in FIG. 13, a thumb tip pressure portion 360 has attached thereto a thumb tip retaining means 363 adjustable in a similar manner to the finger tip retaining means 323 described above. As with the finger tip retaining means the apparatus of U.S. Pat. No. 3,605,199 is relevant as a preferred method of attachment and adjustment. In the embodiment shown in FIG. 13, the thumb tip retaining means comprises a flexible band of a plastics material, permanently attached at one end thereof to one side of the thumb tip pressure portion 360 and having at the other end an adjustment member in the form of a stem 367 with a number of teeth thereon. A receiving aperture 361 is provided at a second side of the thumb tip pressure portion 360 and is adapted to allow passage of the stem 367 therethrough in a first direction but restrict passage of the stem in the second direction by engagement with teeth 368. In this way the stem which constitutes the free end of the thumb tip retaining means 363 can be secured to the thumb tip pressure portion in order to form a loop suitable for retaining a thumb on a thumb tip receiving area (not shown) of the thumb tip pressure portion 360. An inner surface of the loop formed by the thumb tip retaining means constitutes a thumb tip retaining surface 365.

FIGS. 14(a), 14(b), 15(a) and 15(b) show a plunger flange receiving portion in the form of a plunger flange cap 312 defining a cavity 313 therein and a means of attaching the plunger flange cap 312 to a first connection portion in the form of a rod portion 315 as an alternative to the embodiment described with reference to FIGS. 1 to 12. In this embodiment, the plunger flange cap 312 has a first arm 314a and a second arm 314b which together define the edges of a female connection portion adapted for attachment to a first connection portion in the form of a rod portion 315 of a plunger coupling element 310. The female connection portion comprises a recess 314 having three side walls 327a, 328, 329 which are provided with a raised step 311, projecting into the recess 314, thereon. The rod portion 315 has a generally T-shaped cross-section and includes a recess 316 in a wider part 315a thereof, at an end thereof distal from the finger tip pressure portion 320. The recess extends around the three upper most edges (as shown in FIG. 14b) of the wider part 315a and is of an axial length sufficient to accommodate the axial length of the plunger flange cap 312. The wider part 15a of rod portion 15 includes, in the recess 316, a doubly recessed portion 317 which is smaller in axial extent than the recess 316 but otherwise generally follows the form of the recess 316. The doubly recessed portion is adapted to receive the step 311. In this embodiment, after the plunger coupling element 310 is attached to barrel coupling element 350, the plunger flange cap 312, which provides a cavity 313 adapted to receive a syringe plunger flange, may be attached to the rod portion 315 by sliding from the free end of the rod portion 315 until the female connection portion engages with the recess 316 in the rod portion 315, preventing further axial movement of the plunger flange cap 312 relative to the rod portion 315. When in this position, the step 311 engages and is retained in the doubly recessed portion 317 on the rod portion 315, providing additional stability for the connection. If required, additional fixing means such as adhesive or a plastic welding compound may be applied to secure the connection.

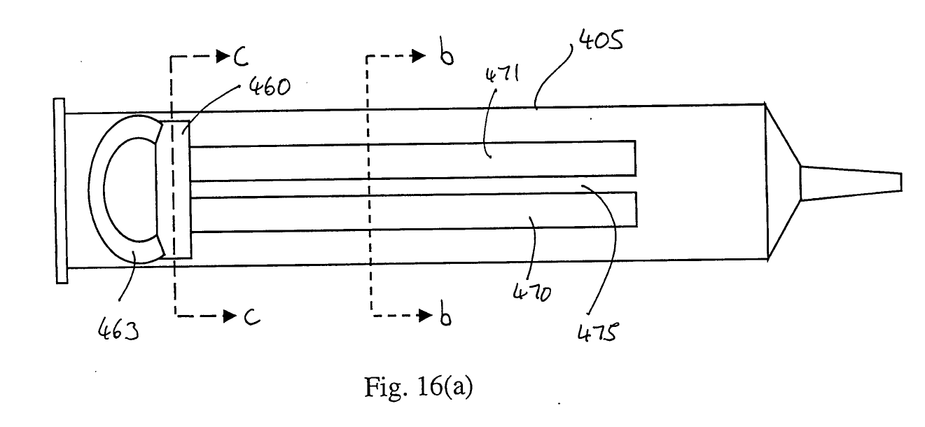

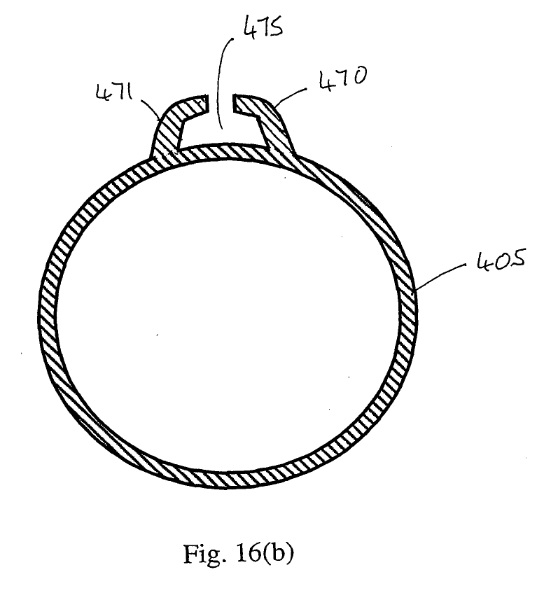

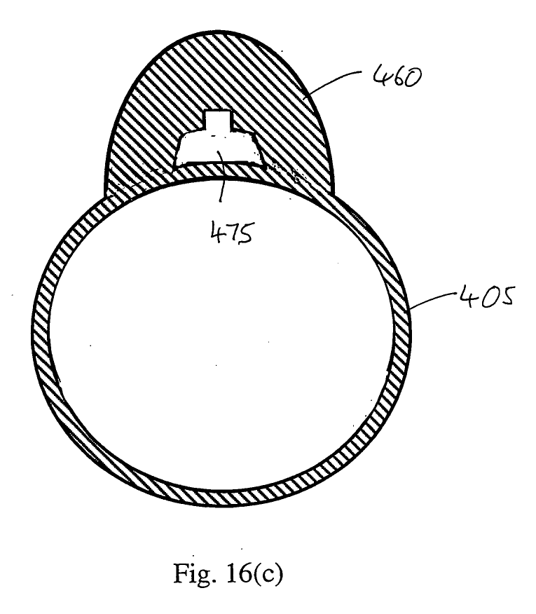

The above two described embodiments are aspiration devices in the form of holders for syringes. In an alternative embodiment of the invention, the aspiration device may be formed as a unit which integrally includes a syringe. In one such embodiment, a plunger coupling element may be substantially as described above in relation to the embodiments in the form of syringe holders (albeit, perhaps, with slight modifications), but the barrel coupling element may be formed integrally with the barrel of a syringe. (It will be understood that the term syringe is used herein to denote both syringes per se and syringe-like portions of aspiration devices.) Such an embodiment is shown in plan view in FIG. 16(a) with schematic cross-sections shown in FIGS. 16(b) and 16(c). In this embodiment, a part which acts as a syringe barrel 405 may include, integrally formed therewith, first and second axially extending channel forming members 470, 471 in the form of a pair of generally parallel, axially extending, elongate rails. These define an axially extending channel 475 suitable for retaining, in sliding relation thereto, a connection rod (of a plunger coupling element) having a generally T-shaped cross-section. Each of the channel forming members includes a portion extending away from the barrel which defines the lateral extent of a wider portion of the channel (which corresponds to the cross bar of the T-shape), and a portion, further from the barrel, extending generally towards, but spaced apart from, the other channel forming member, which defines the lateral extent of a narrow portion of the channel and prevents the connection rod from exiting the channel in the radial direction relative to the barrel. The channel forming members may be regarded as together comprising a barrel coupling element in this embodiment.

A thumb tip pressure portion 460 is also formed integrally with the barrel 405, preferably at the end of the first and second channel forming members 470, 471 which is further from the needle end of the barrel. The thumb tip pressure portion 460 extends away from the central axis of the barrel 405 and is formed generally in a plane perpendicular thereto. The channel 475 extends through the thumb tip pressure portion 460, in order to allow a connection rod of a plunger coupling element (not shown) to pass therethrough. A thumb tip retaining means 463 is also provided. In the embodiment of FIG. 16(a), the thumb tip retaining means shown is similar to the thumb tip retaining means 63 of the embodiment of FIGS. 1 to 12, but it will be understood that other forms of thumb tip retaining means, including but not limited to adjustable thumb tip retaining means, could be incorporated. For example, thumb tip retaining means (and also finger tip retaining means) could include any desired means of keeping the thumb tip (and/or finger tip) in proximity of the device while applying a force in the opposite direction to that applied by pressure of the front of the thumb tip (or finger tip) to the respective pressure portion. Variations could include, but are not limited to, cup-like or thimble-like portions for retaining the thumb (or finger) tip, resilient and/or flexible bands, sleeves or straps attached to (or around) the pressure portions, or, if desired and appropriate, gloves or partial gloves including finger portions attached to the pressure portions, or even hook and loop fasteners or adhesive in a form suitable for temporarily securing thumb (or finger) tips to the pressure portions. It will be appreciated that the barrel 405 and parts formed integrally therewith perform the functions of the barrel coupling elements of the embodiments of FIGS. 1 to 13.

FIGS. 16(b) and 16(c) should be regarded as schematic and not to scale, as the thickness of the barrel wall is exaggerated for clarity. In practice, certain parts of the barrel might have to be thickened or reinforced, compared to normal syringe barrels, in order to accommodate the stresses associated with operation of the device, for example, application of pressure to the thumb tip pressure portion.

Although some preferred embodiments are described above, it should be appreciated that many other embodiments and variations are possible without departing from the scope of the invention. Embodiments suitable for various sizes of syringe are envisaged, and many of their requirements will be ascertainable by the skilled person. In particular, embodiments including or for use with syringes with capacities including, but not limited to 2 ml to 50 ml are envisaged. Injection moulding is a preferred method of manufacture for disposable aspiration devices made from plastics materials.

Reference is made herein to finger tips and thumb tips. For clarity, a finger tip of a particular finger is considered to be that part of a finger between the finger joint which is furthest from the palm of the hand (when the finger is straight) and the end of the finger. A thumb tip is the corresponding part of a thumb. The back of a finger tip (or thumb tip) is considered to be the face of the finger tip (or thumb tip) which normally bears the nail.

The words “comprising”, “having”, and “including” should be interpreted in an inclusive sense, meaning that additional features may also be added.

It is to be understood that, if any prior art publication is referred to herein, such reference does not constitute an admission that the publication forms a part of the common general knowledge in the art, in Australia or in any other country.

Variations and modifications can be made in respect of the invention described above.

1. An aspiration device comprising:
a syringe barrel coupling element, adapted in use to be coupled to a barrel of a syringe, and to remain substantially fixed relative to said barrel; and
a plunger coupling element, adapted in use to be coupled to a plunger of a syringe and to move relative to said barrel coupling element so that forced relative motion of said plunger coupling element and said barrel coupling element forces relative motion of the syringe plunger and syringe barrel;
wherein the plunger coupling element and the barrel coupling element are moveable relative to each other by application of force to respective pressure portions associated with the respective elements;
wherein a first pressure portion, associated with the plunger coupling element, includes two finger tip receiving areas adapted respectively to receive and be forced by an index-finger finger tip and a second-finger finger tip of a user and said finger tip receiving areas have centres which are, in use, substantially equidistant from a longitudinal axis of said syringe;
wherein said pressure portions associated with the elements are adapted to be forced towards each other by a user placing an index-finger finger tip and a second-finger finger tip on the first pressure portion and a thumb tip on a second of said pressure portion; and
wherein forcing said first and second pressure portions generally towards each other forces the plunger to move substantially axially in an outward direction relative to the barrel, and
wherein the first pressure portion and the second pressure portion are arranged so that in use an imaginary line substantially parallel to the longitudinal axis of the syringe passes through the second pressure portion and between the finger tip receiving areas of the first pressure portion.

2. An aspiration device as claimed in claim 1, wherein the barrel coupling element is formed integrally with a barrel of a syringe.

3. An aspiration device as claimed in claim 1, wherein the aspiration device is a holder for a syringe.

4. An aspiration device as claimed in claim 1, wherein said first pressure portion is substantially rigidly coupled to the plunger coupling element.

5. An aspiration device as claimed in claim 1, wherein said first pressure portion constitutes part of the plunger coupling element.

6. An aspiration device as claimed in claim 1, wherein said second pressure portion is substantially rigidly coupled to the barrel coupling element.

7. An aspiration device as claimed in claim 1, wherein said second pressure portion constitutes part of the barrel coupling element.

8. An aspiration device as claimed in claim 1, wherein said second pressure portion is provided with thumb tip retaining means to retain a thumb tip of a user.

9. An aspiration device as claimed in claim 8, wherein said thumb tip retaining means provides a retaining surface against which, in use, the back of the thumb tip may press.

10. An aspiration device as claimed in claim 8 wherein said first pressure portion is provided with finger tip retaining means to retain one or more finger tips of a user.

11. An aspiration device as claimed in claim 10, wherein said finger tip retaining means provides a retaining surface against which, in use, one or more backs of the one or more finger tips may press.

12. An aspiration device as claimed in claim 11, wherein said thumb tip retaining means provides a retaining surface against which, in use, the back of the thumb tip may press, and wherein the device is adapted to allow a user to move said first and second pressure portions away from each other by pressing the back of the thumb tip against the retaining surface of the thumb tip retaining means and the back or backs of the one or more finger tips against the retaining surface of the finger tip retaining means and moving apart said thumb tip from said one or more finger tips.

13. An aspiration device as claimed in claim 10, wherein said finger tip retaining means may be adjusted to allow retention of finger tips of different sizes.

14. An aspiration device as claimed in claim 13, wherein the finger tip retaining means may be adjusted by adjustment means comprising an adjustment member which is selectively locatable in adjustment member retaining means coupled to the pressure portion.

15. An aspiration device as claimed in claim 14, wherein said adjustment member of the finger tip retaining means includes a number of teeth spaced along the length thereof and said adjustment member retaining means includes a pawl portion adapted to engage one or more of said teeth.

16. An aspiration device as claimed in claim 8, wherein said thumb tip retaining means may be adjusted to allow retention of thumb tips of different sizes.

17. ) An aspiration device as claimed in claim 1, wherein said plunger coupling element includes a plunger flange receiving portion and includes a first connection portion between the plunger flange receiving portion and the pressure portion of the plunger coupling element.

18. An aspiration device as claimed in claim 1, wherein said barrel coupling element includes a barrel flange receiving portion, and includes a second connection portion between the barrel flange receiving portion and the pressure portion of the barrel coupling element.

19. An aspiration device as claimed in claim 17, wherein said barrel coupling element includes a barrel flange receiving portion, and includes a second connection portion between the barrel flange receiving portion and the pressure portion of the barrel coupling element, and wherein said first and second connection portions are shaped so that, in use, the plunger coupling element and barrel coupling element are slidably coupled.

20. An aspiration device as claimed in claim 19, wherein, in use, at least part of one of the connection portions is retained in at least part of the other connection portion.

21. An aspiration device, in the form of a holder for a syringe, comprising:
a syringe barrel coupling element, adapted in use to be coupled to a barrel of a syringe, and to remain substantially fixed relative to said barrel; and
a plunger coupling element, adapted in use to be coupled to a plunger of a syringe and to move relative to said barrel coupling element so that forced relative motion of said plunger coupling element and said barrel coupling element forces relative motion of the syringe plunger and syringe barrel;
wherein the plunger coupling element and the barrel coupling element are moveable relative to each other by application of force to at least one pressure portion associated with each element;
wherein a first pressure portion includes two finger tip receiving areas adapted respectively to receive and be forced by an index-finger finger tip and a second-finger finger tip of a user and said finger tip receiving areas have centres which are, in use, substantially equidistant from a longitudinal axis of said syringe;
wherein said pressure portions associated with the elements are adapted to be forced towards each other by a user placing an index-finger finger tip and a second-finger finger tip on a first of said pressure portions and a thumb tip on a second of said pressure portions; and
wherein forcing said first and second pressure portions generally towards each other forces the plunger to move substantially axially in an outward direction relative to the barrel.

22. An aspiration device as claimed in claim 21, wherein the first pressure portion and the second pressure portion are arranged so that in use an imaginary line substantially parallel to the longitudinal axis of the syringe passes through both the first and second pressure portions.

23. An aspiration device as claimed in claim 21, wherein said second pressure portion is provided with thumb tip retaining means to retain a thumb tip of a user.

24. An aspiration device as claimed in claim 23, wherein said first pressure portion is provided with finger tip retaining means to retain one or more finger tips of a user.

25. An aspiration device as claimed in claim 23, wherein said first pressure portion is provided with finger tip retaining means to retain one or more finger tips of a user, and wherein the device is adapted to allow a user to move said first and second pressure portions away from each other by pressing the back of the thumb tip against a retaining surface of the thumb tip retaining means and the back or backs of the one or more finger tips against one or more corresponding retaining surfaces of the finger tip retaining means and moving apart said thumb tip from said one or more finger tips.

26. An aspiration device which allows aspiration and injection modes to be operated using a single hand and with the hand being in substantially the same position relative to the device in both aspiration and injection modes, comprising:
a syringe barrel coupling element, adapted in use to be coupled to a barrel of a syringe, and to remain substantially fixed relative to said barrel; and
a plunger coupling element, adapted in use to be coupled to a plunger of a syringe and to move relative to said barrel coupling element so that forced relative motion of said plunger coupling element and said barrel coupling element forces relative motion of the syringe plunger and syringe barrel;
wherein the plunger coupling element and the barrel coupling element are moveable relative to each other by application of force to at least one pressure portion associated with each element; and
said pressure portions associated with the elements are adapted to be forced towards each other by a user placing a finger tip on a first of said pressure portions and a thumb tip on a second of said pressure portions and wherein forcing said first and second pressure portions generally towards each other forces the plunger to move substantially axially in an outward direction relative to the barrel; and
wherein finger tip retaining means is provided to retain the at least one finger tip in relation to the first pressure portion, and a thumb tip retaining means is provided to retain the thumb tip in relation to the second pressure portion, and wherein forcing the first and second pressure portions generally away from each other forces the plunger to move substantially axially in an inward direction relative to the barrel.

27. An aspiration device as claimed in claim 26, wherein said finger tip retaining means provides a retaining surface against which, in use, one or more backs of the one or more finger tips may press.

28. An aspiration device as claimed in claim 26, wherein said thumb tip retaining means provides a retaining surface against which, in use, the back of the thumb tip may press.

29. An aspiration device as claimed in claim 27, wherein said thumb tip retaining means provides a retaining surface against which, in use, the back of the thumb tip may press, and wherein forcing the finger tip pressure portion away from and the thumb tip pressure portion away from each other by pressure of the back of at least one finger tip on said finger tip retaining surface and the back of the thumb on the thumb tip retaining surface, operates the injection mode of the device.

30. An aspiration device as claimed in claim 26, wherein the device is a holder for a syringe.

31. An aspiration device as claimed in claim 26, wherein at least part of the device is formed integrally with a syringe.

32. An aspiration device as claimed in claim 26, wherein said first pressure portion is adapted to receive and be forced by an index-finger finger tip and a second-finger finger tip of a user.

33. An aspiration device as claimed in claim 26, wherein said first pressure portion includes two finger tip receiving areas which have centres which are, in use, substantially equidistant from a longitudinal axis of said device syringe.

34. An aspiration device as claimed in claim 26, wherein said first pressure portion is substantially rigidly coupled to the plunger coupling element.

35. An aspiration device as claimed in claim 26, wherein said first pressure portion constitutes part of the plunger coupling element.

36. An aspiration device as claimed in claim 26, wherein said second pressure portion is substantially rigidly coupled to the barrel coupling element.

37. An aspiration device as claimed in claim 26, wherein said second pressure portion constitutes part of the barrel coupling element.

38. An aspiration device as claimed in claim 26, wherein said first pressure portion is adapted to receive and be forced by an index-finger finger tip of a user.

39. An aspiration device as claimed in claim 38, wherein said first pressure portion is adapted to receive and be forced by an index-finger finger tip and a second-finger finger tip of a user.

40. An aspiration device as claimed in claim 26, wherein said finger tip retaining means is adjustable to allow retention of finger tips of different sizes.

41. An aspiration device as claimed in claim 26, wherein the finger tip retaining means is adjustable by adjustment means comprising an adjustment member which is selectively locatable in adjustment member retaining means coupled to the pressure portion.

42. An aspiration device as claimed in claim 41, wherein said adjustment member of the finger tip retaining means includes a number of teeth spaced along the length thereof and said adjustment member retaining means includes a pawl portion adapted to engage one or more of said teeth.

43. An aspiration device as claimed in claim 26, wherein said thumb tip retaining means is adjustable to allow retention of thumb tips of different sizes.

44. An aspiration device as claimed in claim 26, wherein said plunger coupling element includes a plunger flange receiving portion and includes a first connection portion between the plunger flange receiving portion and the pressure portion of the plunger coupling element.

45. An aspiration device as claimed in claim 26, wherein said barrel coupling element includes a barrel flange receiving portion, and includes a second connection portion between the barrel flange receiving portion and the pressure portion of the barrel coupling element.

46. An aspiration device as claimed in claim 44, wherein said barrel coupling element includes a barrel flange receiving portion, and includes a second connection portion between the barrel flange receiving portion and the pressure portion of the barrel coupling element and wherein said first and second connection portions are shaped so that, in use, the plunger coupling element and barrel coupling element are slidably coupled.

47. An aspiration device as claimed in claim 46, wherein, in use, at least part of one of the connection portions is retained in at least part of the other connection portion.

48. A method of aspirating a fluid comprising:
providing an aspiration device which comprises:
a syringe barrel coupling element coupled to a barrel of a syringe;
a plunger coupling element, coupled to a plunger of a syringe, which is moveable relative to the barrel coupling element,
a first pressure portion associated with the plunger coupling element;.
a second pressure portion associated with the barrel coupling elements;
placing at least one finger tip of a hand on the first pressure portion and a thumb tip of the hand on the second pressure portion;
and forcing the first and second pressure portions towards each other, along an imaginary line which extends between the first and second pressure portions and which is substantially parallel to the axis of said syringe, thereby forcing the plunger to move substantially axially in an outward direction relative to the barrel and effecting aspiration of the fluid.

49. (canceled)
